(19) United States
(12) Patent Application Publication
(10) Pub. No.: US 2012/0164194 A1
(43) Pub. Date: Jun. 28, 2012

(54) Cosmetic Sampler Sheet

(75) Inventors: Leona G. Fleissman, Ridgewood, NJ (US); Tao Zheng, New City, NY (US)

(73) Assignee: AVON PRODUCTS, INC., Suffern, NY (US)

(21) Appl. No.: 13/307,517

(22) Filed: Nov. 30, 2011

Related U.S. Application Data
(60) Provisional application No. 61/426,113, filed on Dec. 22, 2010.

(51) Int. Cl. A61K 8/02; A61K 8/81; A61K 8/84; B05D 3/00; A61K 8/88; B05D 5/00; B05D 3/02; A61K 8/73; A61K 8/89

(52) U.S. Cl. 424/401; 424/63; 427/379

(57) ABSTRACT

A cosmetic sample sheet is provided which includes cosmetics contained within or under a solid, water-soluble polymeric film, and supported by a substrate sheet. The cosmetics are easily reconstitutable by a consumer to allow the consumer to try different shades or colors of particular cosmetics.

### CROSS REFERENCE TO RELATED APPLICATIONS

This application claims priority benefit, under 35 U.S.C. §119(e), of U.S. Provisional Patent Application No. 61/426,113, filed Dec. 22, 2010, the contents of which application are hereby incorporated by reference in their entirety,

### FIELD OF THE INVENTION

The present invention relates generally to cosmetic compositions comprising a solid, continuous matrix of a water-soluble polymer, and in particular to such cosmetic formulations which can be reconstituted with water prior to use to provide a liquid cosmetic. The invention also relates to cosmetic samplers.

### BACKGROUND

Cosmetic samplers are widely employed at stores so that customers may assess a variety of hues of color cosmetics before making a purchase. Individual squares having a powdered cosmetic, such as eye shadow or blush, deposited on a plastic substrate and covered with a protective mask are known. However, many cosmetic products, including foundations, mascara, concealers, lip glosses, nail polishes, etc., exist in liquid form and do not lend themselves to that type of sampler. Liquid cosmetics are typically contained in glass or plastic containers to prevent spillage and to facilitate dispensing for application to skin or other desired area. These products are typically sampled at department stores in the bottles in which they are sold and samples are repeatedly drawn from the same containers for several customers to try. Not only does this raise hygienic concerns, but it limits the channels through which samples of liquid cosmetics can be distributed. For example, it is not currently practical to distribute liquid cosmetic samples in mailers, magazines, or the like.

What is lacking is a convenient form for delivering liquid cosmetics. Dissolvable films have been used in other industries to deliver liquid components, the most notable example being the Listerine® Breath Strips. These types of products comprise a water-soluble polymeric matrix composed of a natural or synthetic polymer such as pultulan. To date, there have been no successful efforts to develop cosmetic products based on this technology.

It is therefore an object of the present invention to provide convenient forms for distributing samples of cosmetics, in particular liquid cosmetics, which can be reconstituted by addition of water before or during use. It is a further object of the invention to provide cosmetic compositions comprising one or more cosmetic ingredients, such as colorants or therapeutic actives, dispersed in a matrix of water-soluble polymer such that the composition is in solid form but is readily reconstitutable with water.

### SUMMARY OF THE INVENTION

In accordance with the foregoing objectives and others, the present invention provides novel forms for delivering cosmetic compositions to consumers. The advantages of the invention will be most fully realized where the intended cosmetic is one that is traditionally in liquid form, as is the case with many foundations, concealers, lip glosses, and nail polishes. Such liquid cosmetics are difficult to distribute to consumers as samples in magazines, catalogues, and mailers, and no satisfactory solution exists for providing samples of these liquid cosmetics at retailers. However, the invention is not limited to samplers and includes, in its broadest aspect, novel cosmetic forms that will improve the convenience and portability of cosmetics.

In one aspect of the invention, a cosmetic product, including but not limited to a cosmetic sampler, is provided. The cosmetic product includes a sheet-like substrate formed of plastic or paper having a cosmetic formulation deposited on at least a portion of the substrate. Typically, the cosmetic formulation is dry or semisolid because any volatiles have been removed after it is deposited onto the substrate. A layer of a solid water-soluble polymer forms a continuous layer over at least the portion of the substrate that bears the cosmetic formulation to protect the cosmetic formulation from transfer. Alternatively, the cosmetic formulation can be included with the dry water-soluble polymer such that it is homogenously dispersed throughout a matrix formed by the water-soluble polymer. In either case, the layer of dry, water-soluble polymer may be overlaid with a masking layer of tissue or thin plastic, for example, to protect the composition prior to use. In use, the consumer mixes an amount of water to the product sufficient to dissolve the water-soluble polymer and reconstitute the liquid cosmetic.

In another aspect of the invention, a cosmetic sampler sheet is provided. The sampler sheet will have a plurality of sampling areas on a plastic or paper substrate in the form of a sheet and a cosmetic formulation is deposited on each of the sampling areas. In some implementations, the cosmetic formulations will be different at each sample area such that the sample sheet can provide a variety of cosmetics of different shade or other optical attribute. A water-soluble polymeric matrix forms a solid layer over each of the sampling areas and protective mask may be provided to protect the water-soluble polymeric layer. The cosmetic formulations can be reconstituted with a suitable volume of water to produce a liquid cosmetic composition for application to a human integument, such as skin, hair, lashes, eyebrows, and nails. The substrate is typically perforated or scored to permit each sampling area to be readily separated from the others. The sampler sheets can be included in promotional materials, such as magazine or catalogue inserts or as mailers, or may be distributed at retail locations.

Also provided is a method of making a cosmetic sample sheet comprising providing a substrate formed of a sheet of plastic or paper having a plurality of sampling areas defined thereon and depositing cosmetic formulations on each of the sampling areas. Typically, though not necessarily, the cosmetic formulations will be different at each sampling area and will be applied simultaneously to the sheet with multiple filling heads. The cosmetic formulations will typically comprise volatile constituents, such as water, and thus the substrate will be heated to remove the volatile components. After the volatiles are removed from the cosmetic composition to leave a residue thereof on the substrate, a solution or dispersion of water-soluble polymer is applied, typically by spraying, over said sampling areas to cover at least the residue of the cosmetic composition. The sample sheet is then heated to dry and solidify the water-soluble polymer into a coating layer covering the cosmetic formulations. The substrate is typically perforated or scored to permit each sampling area to be readily separated from the others.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### BRIEF DESCRIPTION OF THE DRAWINGS

FIG. 1 illustrates a person care product 10 according to the invention which comprises a substrate 20, a topical formulation 40 disposed on the substrate, a solid film of water soluble polymer 30 overlaying the substrate and topical formulation, and a removable masking film 50 over the layer of water-soluble polymer 30.

FIG. 1A is a cross-sectional view of the personal care product 10 of FIG. 1.

FIG. 2 illustrates a cosmetic sampler 100 according to the invention which comprises a substrate 120 having perforations 170 which define individual sampling areas, each sampling area comprising a cosmetic formulation 140A-L, each of which may be the same of different, disposed on the substrate 120, a solid film of water soluble polymer 130 overlaying the substrate and cosmetic formulations 140A-L, and a removable masking film 150 over the layer of water-soluble polymer 130.

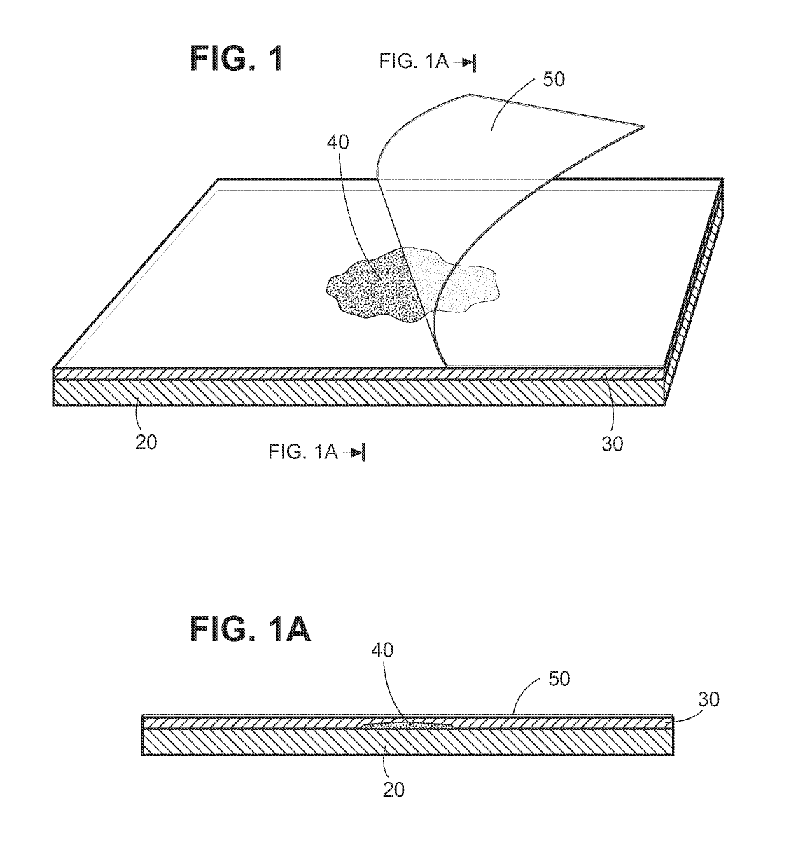

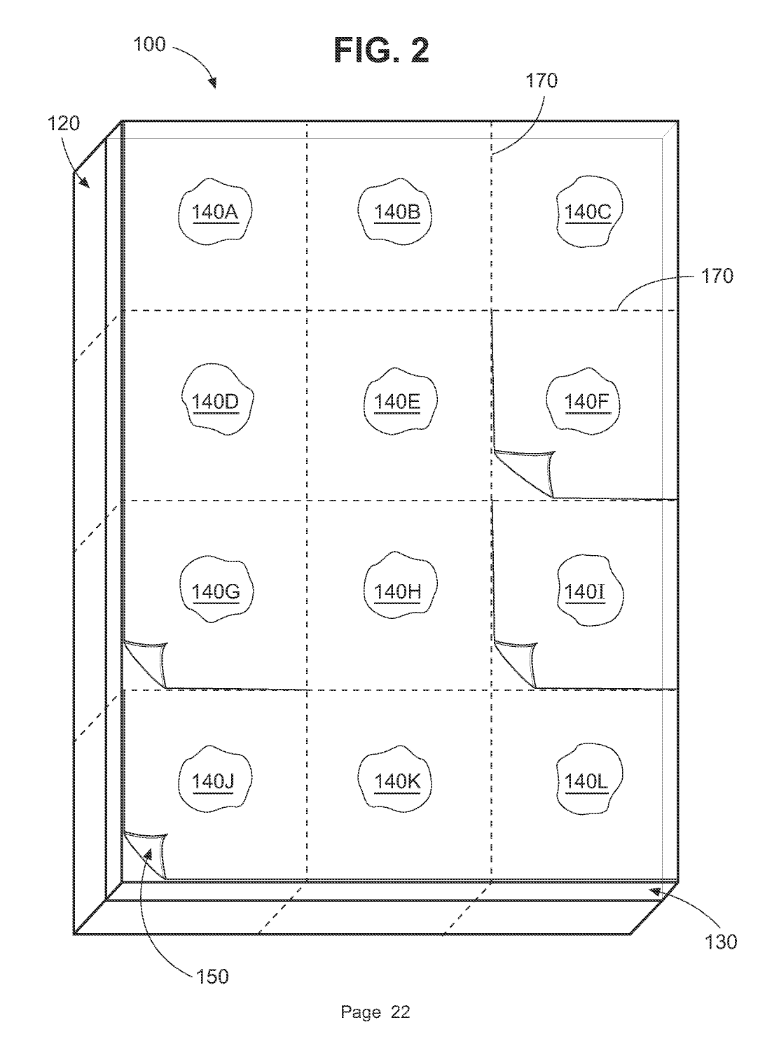

### DETAILED DESCRIPTION

The invention concerns personal care products provided in novel forms. As used herein, the term “personal care product” refers to compositions that are intended to be topically applied to a person. Examples of personal care compositions include skin care products, cosmetics, sunscreens, and the like. In preferred embodiments, the personal care products are cosmetics, and in particular color cosmetics. As used herein, the term “consisting essentially of” is intended to limit the invention to the specified materials or steps and those that do not materially affect the basic and novel characteristics of the claimed invention, as understood from a reading of this specification.

The personal care products of the invention include a substrate composed of plastic or paper (cellulosics). The substrate takes the form of a sheet by which is mean that it is elongated in two dimensions but is essentially flat, having a thickness between about 0.1 and about 20 mils (the term “mil” refers to 1/1000 of an inch). The sheet may, for example, have one dimension that is from about ½ inch to about 36 inches long, a second dimension that is from about ½ inch to about 36 inches long, and a typical thickness from about ½ mils to about 10 mils. More typically, the sheet will have one dimension that is from about one inch to about 12 inches long, a second dimension that is from about one inch to about 12 inches long, and a typical thickness from about one mil to about 5 mils. The sheet will ideally be flexible and sufficiently resistant to water and other volatile solvents, such as alcohols, lower hydrocarbons, and silicone fluids, such that it is not deformed or damaged when a liquid cosmetic is applied to its surface. The sheet will ideally also be able to withstand heating in an oven to temperatures required to eliminate volatile solvents, which will depend of course on the specifics of the formulation but will usually comprise heating in an oven to a temperature between about 50° C. and about 100° C., more typically, about 60° C. and about 80° C.

In some embodiments, a topical formulation is deposited on at least a portion of the substrate. The topical formulation will be one used for personal care, most commonly a cosmetic, that is topically acceptable. “Topically acceptable” refers to ingredients that are generally regarded as safe for application to a human integument, including hair of the scalp, eyelashes, eyebrows, skin of the face, tips, nails, etc. The topically acceptable ingredients will include, without limitation, colorants (pigments, takes, and dyes), fillers, film formers (water-resistant and/or oil resistant), emollients, humectants, emulsifiers, thickeners, rheology modifiers, chelating agents, pH adjusters, skin conditioning agents, active (therapeutic) agents, sunscreens, fragrances, preservatives, and the like. Any ingredient that is customarily found in the particular topical formulation is contemplated to be suitable.

The preferred personal care products are cosmetic compositions, and in particular color cosmetics which, by definition, comprise colorants. The colorants may comprise from about 5% to about 99% by weight of the residue of the composition after volatiles have been removed, more typically, from about 20% to about 98% by weight of the residue. The colorants may be pigments, lakes, or dyes. There is essentially no limitation on the nature of the pigments, lakes, or dyes and include those listed in the International Cosmetic Ingredient Dictionary and Handbook, 12^{th} Ed. (2008), incorporated by reference herein.

The term “pigment” refers to an inorganic or organic particle that is insoluble in the aqueous base of the intended cosmetic and is intended to impart color or hiding power. Preferred pigments are inorganic pigments, such as metal oxides and hydroxides, particular mention being made of magnesium oxide, magnesium hydroxide, calcium oxide, calcium hydroxides, aluminum oxide, aluminum hydroxide, chromium oxide, chromium hydroxide, cobalt oxide, iron oxides, iron hydroxides manganese oxides, nickel oxide, tin oxide, titanium dioxide, zirconium oxide, and zinc oxide to name but a few. Other suitable pigments include ultramarine blue (i.e., sodium aluminum silicate containing sulfur), Prussian blue, manganese violet, sericite, potassium ferricyanide, potassium ferrcyanide, potassium ferrocyanide trihydrate, magnesium carbonate, calcium carbonate, silica, talc, mica, magnesium silicate, aluminum magnesium silicate, carbon black, and composite oxides and composite hydroxides such as iron titanate, cobalt titanate and cobalt aluminate to name a few. For the purposes of this invention, fillers which impart hiding power, including talc, calcium carbonate, and silica, are also considered pigments. The pigments may he surface-modified with hydrophobic or hydrophilic coatings to enhance or alter one or more properties such as their optical properties, dispersibility, or substantivity with the integument, examples including the COVALUMINE™ line of pigments marketed by SENSIENT™ Cosmetic Technologies LCW which comprise pigments bonded to a platelet-shaped alumina substrate and a triethoxycaprylylsilane surface treatment covering the particle at about 1% by weight, the AEROSIL™ line of hydrophobically modified finned silica particles from Degussa Corporation of Parsippany, N.J., and the AEROXIDE™ ALU C805 hydrophobically modified alumina from Evonik Industries.

The topical formulations may also comprise a pearling agent, alone or in combination with pigment and other colorants. The pearling agents will typically be titanated micas or bismuth oxychloride. The titanated micas can be white or colored. Colored titanated micas will have iron oxides or other pigments deposited thereon to alter the hue of the particle. Bismuth oxychloride pearls may also be bonded to colored pigments to vary the hue, as in the CHROMA-LITE™ line from Englehard Corporation (Iselin, N.J.), which is a combination of a colored pigment bonded to bismuth oxychloride and mica using calcium stearate. The pearl can also include a natural nacre. Also suitable are terephthalates to produce glitter, e,g. polyethylene terephthalate.

The topical formulations can also comprise other particulates including, without limitation, kaolin, Nylon powders such as Orgasol™, polyethylene powder, Teflon™ powder, starch, boron nitride, cross-linked polymethacrylate copolymers powders such as Polytrap™ (Dow Corning), polymeric microspheres such as Expancel™ (Nobel Industries), silicone resins microbeads such as Tospearl™ from Toshiba), and combinations thereof. Other suitable particulates include the particulate silicon wax sold under the trade name Tegotop™ 105 (Degussa/Goldschmidt Chemical Corporation) and the particulate vinyl polymer sold under the name Mincor™ 300 (BASF).

The topical formulations may include non-volatile solvents and oils including without limitation, fatty alcohols, fatty acid esters, lanolin, hydrocarbon oils, silicone oils, and mixtures thereof. The topical formulation may also include film forming agents selected from the group consisting of waxes, water-soluble polymeric film formers, and water-insoluble polymeric film formers. “Film forming agents” are well known and comprise a group of chemicals that leave a pliable, cohesive, and continuous covering over an integument when applied on its surface. In other embodiments, the topical compositions will be free of wax and/or free of water-insoluble polymeric film formers. While the topical formulations may, in some embodiments, include emollients and humectants, in other embodiments, they will be free of emollients and/or humectants (including glycerin).

The topical formulations may comprise one or more volatile solvents to facilitate their application to the substrate through fill nozzles, droppers, or other expedient for meting the formulation onto an area of the substrate. The volatile solvents may be, without limitation, water, C₃-C₆ ketones (e.g., acetone), C₃-C₆ esters (e.g., ethyl acetate), C₂-C₆ alcohols (e.g., ethanol), silicones (e.g., trimethicone, cyclomethicone pentamer, etc.), hydrocarbons (e.g., isododecane), or combinations thereof. Because the volatiles are removed after the topical formulation is applied to the substrate, their identity is not critical. The topical formulation may be applied to a specific area of the substrate, or may cover the major portion of a side of the substrate, or may cover an entire side of the substrate.

After the topical formulation is deposited onto the substrate, it is dried to remove any volatiles. The drying may be carried out at room temperature but preferably is carried out at elevated temperature to accelerate the removal of volatiles. Usually, the drying is performed in a convection oven at a temperature between about 30° C. and about 120° C., more typically between about between about 50° C. and about 90° C., and preferably between about 60° C. and about 80° C. The drying is carried out for a time sufficient to substantially remove the volatiles and to reduce the formulation to a solid or semi-solid residue on the substrate.

A layer of a water-soluble polymer is then deposited over at least a portion of the substrate, including the portion on which the residue of the topical formulation is deposited. The term “water-soluble polymer” includes polymers with a solubility, measured at 25° C., of at least 0.1 gram/liter (g/L), but preferably at least 1.0 g/L. The water-soluble polymers should be topically acceptable, as they will ultimately be dissolved and included in the final formulation that is applied to a human integument.

The water-soluble polymers may be synthetic, natural, or modified polymers derived from natural polymers. Representative water-soluble polymers include wheat or soybean proteins; keratin hydrosylates and sulfonic keratins; casein; albumin; agarose; collagen; glutelin; glucagon; gluten; zein; gelatins and derivatives thereof; polymers derived from chitin or from chitosan, such as anionic, cationic, amphoteric or nonionic chitin or chitosan polymers; polysaccharide polymers such as cellulose-based polymers, for instance cellulose acetate, cellulose acetate phthalate, hydroxyethylcellulose, hydroxypropylcellulose, hydroxymethylcellulose, methylcellulose, ethylhydroxyethylcellulose, hydroxypropylmethylcellulose, hydroxylpropylmethylcellulose phthalate, carboxymethylcellulose and quaternized cellulose derivatives, starches and derivatives thereof, amylose, high amylose starch, hydroxypropylated high amylose starch, and dextrans; acrylic polymers or copolymers such as polyacrylates, polymethacrylates and copolymers thereof; acrylamides such as poly(2-acrylamido-2-methyl-1-propanesulfonic acid); vinyl polymers such as polyvinylpyrrolidones and cross-linked povidone, copolymers of methyl vinyl ether and of maleic anhydride, the copolymer of vinyl acetate and crotonic acid, copolymers of vinylpyrrolidone and of vinyl acetate, copolymers of vinylpyrrolidone and caprolactam, and polyvinyl alcohols; vegetable gums and other natural hydrocolloids such as gum arabic, guar gum, tragacanth gum, xanthan gum and derivatives or karaya gum, alginates, such as sodium alginate, carrageenans, pectin, ulvanes and other algal colloids, pullulan, pectin, mannan and galactomannans, glucomannans; mucopolysaccharides including glycoaminoglycans, hyaluronic acid and its derivatives, and chondroitin sulphate; shellac; sandarac gum; dammar resins; elemi gums; copal resins; caprolactams; poloxamer; water soluble waxes, and mixtures and/or derivatives thereof.

Particular mention may be made of modified celluloses, polyacrylic acid and copolymers, polyacrylamides, polyvinyl alcohol and copolymers, vinyl alcohol/vinyl acetate copolymers, polyvinyl pyrrolidone, gelatin, pullan, gaur gum, xanthan gum, gum acacia, polyethylene oxide, natural or synthetic hydrogels, and combinations thereof. Preferably, the water-soluble polymer comprises a cellulosic, such as cellulose esters, alkyl ethers of cellulose, hydroxyalkyl ethers of cellulose, and carboxyalkyl ethers of cellulose. Specific mention may be made of cellulose acetate, cellulose triacetate, cellulose acetate phthalate, hydroxymethylcellulose, hydroxyethylcellulose, hydroxypropylcellulose, methylcellulose, ethylhydroxy ethyl cellulose, hydroxyethyl methylcellulose, hydroxypropylmethylcellulose, hydroxylpropylmethylcellulose phthalate, carboxymethylcellulose, quaternized cellulose derivatives, and combinations thereof. In one embodiment, the water-soluble polymer is a cellulose ether based polymer, polyethylene oxide, or mixtures thereof. In one embodiment, the water-soluble polymer is at least one of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose.

The water-soluble polymer is preferably dissolved or dispersed in a suitable volatile solvent to aid in application to the substrate. The volatile solvent may be any solvent that is capable of dissolving or dispersing the water-soluble polymer, but should be one that is topically acceptable in the event that residual amounts remain after drying. The solvent for the water-soluble polymer may be non-polar, polar aprotic, or polar protic, and may be water-miscible or water-immiscible, and includes without limitation, water; acetic acid; ethers such as dimethyl ether, diethyl ether; straight or branched chain alcohols, including methanol, ethanol, propanol, isopropanol and butanol; ketones such as acetone or methyl ethyl ketone; esters such as ethyl acetate; siloxanes; hydrocarbons; toluene, benzene, acetonitrile, dimethylformamide; dimethylsulfoxide; tetrahydrofuran; dichloromethane or chloroform; and combinations thereof. Preferably, the solvent is aqueous and may comprise water or consist essentially of water, by which is meant that other solvents are not intentionally added at level that would measurably affect the vapor pressure or flash point of the solvent system.

Once dissolved or dispersed in the volatile solvent, the water-soluble polymer will comprise from about 0.1% to about 95% of the total weight of the combination, more typically from about 10% to about 70% by weight.

To the dispersion or solution of water-soluble polymer may also be added other agents to modify a characteristic of the resulting matrix, such as its hardness or dissolution time. Suitable thickeners include those described in U.S. Patent Pub. 2010/0204341, to Yu et al., the disclosure of which is hereby incorporated by reference. These include, without limitation, carboxyvinyl polymers, such as the products sold under the names CARBOPOL and PEMULEN (INCI name: Acrylates/C₁₀₋₃₀ acrylate crosspolymer; available from Noveon), polyacryiates & polymethacrylates, such as the products sold under the names LUBRAJEL and NORGEL (from Guardian) or HISPAGEL (from Hispano Chimica), polyacrylamides, and sodium polyacrylates/dimethicone/cyclopentasiloxane/tri-deceth-6/PEG-PPG-18/18 dimethicone, polyacrylamides, for example, polyacrylamide/C13-C14 isoparaffin/laureth-72-acrylamido-2-methylpropanesulfonic acid polymers and copolymers, poly(2-acrylamido-2-methylpropane-sulfonic acid) sold by Clariant (INCI name: ammonium polyacryldimethyltauramide), emulsified crosslinked anionic copolymers of acrylamide and AMPS, such as those sold under the name SEPIGEL 305 (INCI name: Polyacrylamide/C13-14 Isoparaffin/Laureth-7; from Seppic) and under the name SIMULGEL 600 (INCI name: Acrylamide/Sodium acryloyldimethyltaurate copolymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, starches, associative polymers, associative polyurethanes, copolymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (from Huls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (from Rohm & Haas), water-soluble vinyl polymer, and celluloses such as microcrystalline cellulose, carboxymethylcellulose, hydroxymethylcellulose and hydroxypropylcellulose. Thickeners, if present, will typically comprise from about 0.01% to about 50% by weight based on the weight of the water-soluble film-forming polymer. Jojoba hydrate, which is a mixture of jojoba alcohol, potassium jojobate, and butylene glycol from Desert Whale, provides good aesthetic properties to the film.

Plasticizers may also be added, including, without limitation trialkyl citrates, polyols, such as glycerin, propylene glycol, sorbitol, dipropylene glycol, butylenen glycol, pentylene glycol, caprylyl glycol, polyethylene glycols, such as PEG 400, mono-and di-saccharides, and polyetheyleneoxide, to name a few. Propylene glycol is a preferred plasticizer. The plasticizer, if present, will typically comprise from about 0.01% to about 50% by weight based on the weight of the water-soluble film-forming polymer.

Any other components typically associated with the intended cosmetic can also be added to the water-soluble polymeric solution or suspension, provided that these additional components do not adversely affect the solid film formed thereby. These include, without limitation, any of the detackifiers, conditioners, and emollients listed in U.S. Patent Pub, 2010/0204341, to Yu et al., the disclosure of which is hereby incorporated by reference; as well as colorants, preservatives, pH adjustors, reducing agents, fragrances, foaming agents, tanning agents, depilatory agents, flavors, astringents, antiseptics, deodorants, antiperspirants, insect repellents, sunscreens, sun blocks, bleaches and lighteners, anti-dandruff agents, adhesives, polishes, strengtheners, fillers, barrier materials, and biocides.

In one alternative embodiment, the topical formulation is not deposited directly onto the substrate, but rather is homogenously dispersed throughout the water-soluble polymer matrix. In that case, the topical formulation may be combined with the solution or suspension of the water-soluble polymer prior to spraying it or otherwise applying it to the substrate.

The suspension or solution of water-soluble polymer, optionally comprising a thickener, plasticizer, etc., is than applied to the surface of the substrate to which the topical formulation was applied. The application should cover at least that portion of the substrate bearing the residue of the topical formulation, but may cover the entire surface of the substrate on that side. The solution or suspension may be cast, painted, sprayed, extruded, enrobed or otherwise deposited onto the surface, for example by dipping the substrate into the solution or suspension, in the preferred practice the solution or suspension is sprayed onto the surface through a pressurized nozzle. The amount of material applied should be sufficient to provide a film, on hardening of about 0.1 mils to about 20 mils in thickness.

After the water-soluble polymer has been applied, the volatiles are removed by evaporation or by heating to solidify the water-soluble polymer into a solid film over the substrate, such that the residue of the topical formulation will be sandwiched between the substrate and the water-soluble polymeric film. The volatiles are preferably removed by heating to a temperature sufficient to accelerate removal of volatiles but low enough so as not damage the substrate or film. In one embodiment, the sheet is heated to a temperature between about 40° C. and about 150° C., preferably between about 50° C. and about 90° C., and more preferred still from about 60° C. to about 80° C. (e,g., ˜70° C.).

A masking layer of tissue or plastic may be place over top of the water-soluble polymeric film to protect the film until the product is ready for use, although its use is strictly optional. The masking layer, if used, should be readily pealable from the water-soluble polymer layer and will ideally be transparent so that the colors of the cosmetics can be seen.

In use, the consumer will add water to the water-soluble film, for example by applying water drop-wise onto the surface, to dissolve the film. The film should be capable of dissolving in less than 30 seconds, preferably less than 20 seconds, and more preferably, in less than 10 seconds according to the dissolution test disclosed in U.S. Patent Pub, 2010/0204341, to Yu et at., the disclosure of which is hereby incorporated by reference. The user will thoroughly mix the water and film to dissolve it and to homogenize the residue of the topical formulation with the water and film. In this manner, the topical formulation is reconstituted as a liquid formulation for topical application to a human integument. The composition may be mixed with the fingers, an applicator, or the like. Typically, the topical formulation will be one that can be reconstituted to form a cosmetic, and in particular, a liquid cosmetic, including without limitation, a foundation, concealer, mascara, nail polish, or lip gloss.

In a preferred embodiment, the topical formulation is a foundation comprising pigment and/or pearlescents. In one embodiment, the sheet will be from 1 to 10 inches long, from 1 to 10 inches wide, with a thickness of between 0.1 and about 20 mils, and will have a layer of water soluble polymer thereon having a thickness from 0.1 mil to about 20 mils, and optionally will be provided with instructions for the user to add from 1 to about 25 drops of water to the surface, more typically from about 1 to about 10 drops, and preferably from about 1 to about 5 drops of water, to dissolve the film. The cosmetic will be mixed until homogenous and then applied to the skin of the face.

In one embodiment of the invention, the personal care product is a sheet for sampling different cosmetics, for example cosmetics of different shade, hue, lightness, or other optical property. The sheet may be divided into a plurality of different pre-defined sampling areas, which may be separatable from one another by scoring or perforations in the substrate. However, the sheet does not have to be scored or perforated to define the plurality of areas. The sampler sheet will typically have dimensions as described above, and preferably will be suitably dimensioned for inclusion in catalogues, mailers, or the like. The sampling areas will typically be arranged in a grid, such as with rows having from one to 10 squares and columns having from one to 10 squares, but more commonly will be arranged in rows of 2 to 8 squares and columns of 2 to 8 squares.

The sample sheet may be prepared by individually or simultaneously applying different colors/shades of a cosmetic composition on to distinct sampling areas of the sample sheet using different filling heads. The sheet may then heated be heated in an oven as described previously, for example, at approximately 70° C., to dry out the volatiles in the liquid cosmetics and to leave a solid or semi-solid deposit of the cosmetic on the substrate. Alternatively, the cosmetic can be printed onto the substrate using known techniques. A layer of water-soluble polymer is then deposited (typically by spraying) upon the residue of the liquid cosmetic on the sheet. The sheet may then be heated to remove volatile solvents and dry the water-soluble polymers to a hard film.

The sampler sheet of the present invention facilitates direct selling by providing the sample sheets within a catalog and providing multiple squares of foundation shades to the consumer to test, pick and choose. The sampler sheet also affords the opportunity for salespeople to distribute samples to customers in stores or during home sales visits. The sample sheets may be loosely disposed within magazines and other periodicals and/or may be affixed within the magazines to allow purchasers to tear them from the magazine and sample the different shade/color samples on the sheet. In another manner, the sample sheet can be included within a catalog sent to customers, subscribers and/or disseminated by salespeople. The sample sheet allows consumers to try different shades and colors in advance of purchasing. The sheet is portable and thus can also be used by consumers in a “touch-up” manner when traveling or on-the-go. The consumer also has an opportunity to customize a shade by blending two or more shades where multiple sections are provided, each with different shades.

In a preferred embodiment the personal care composition is a liquid cosmetic, and more preferably a cosmetic foundation or cream, or lip gloss or lip coloring composition for application upon the face or skin. The liquid cosmetic will typically be provided on the sample sheet in an array of different shades and colors. The different colors are developed via the inclusion of one or more pigments, pearls, lakes, or dyes.

In a preferred embodiment the sample sheet may be perforated into a plurality of sample squares or quadrants upon each of which a cosmetic sample is deposited. The perforation of the sheet facilitates the easy tearing of a desired sample from the entire sheet. The cosmetic sample may then be peeled from the backing substrate and dissolved or reconstituted with water or water may be added directly to the sheet and mixed in situ on the sheet.

The cosmetic products can take any semisolid or liquid product form suitable for application to the skin, lips, hair, eyelashes, and/or eyebrows, such as a cream, ointment, solution, paste, pomade, gel, lotion, or mascara. Preferred products are foundations, concealers, skin creams, and lipsticks and glosses.

The compositions of the invention may optionally comprise other active and inactive ingredients typically associated with cosmetic and personal care products, including, but not limited to, excipients, fillers, emulsifying agents, antioxidants, surfactants, film formers, chelating agents, gelling agents, thickeners, emollients, humectants, moisturizers, vitamins, sodium ascorbyl/cholesteryl phosphate, minerals, botanicals, viscosity and/or rheology modifiers, sunscreens, keratolytics, depigmenting agents, retinoids, hormonal compounds, alpha-hydroxy acids, trioxaundecanedioic acid, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, staining agents, depigmenting agents, hypopigmenting agents, preservatives, stabilizers, pharmaceutical agents, photostabilizing agents, and mixtures thereof.

The following examples are meant to demonstrate certain aspects of the invention in a non-limiting fashion.

### EXAMPLES

### Example 1

Preparing a Cosmetic Sampler Sheet

Drop different colors/shades of a liquid cosmetic on to distinct areas of a sample sheet simultaneously using different filling heads.

Heat the sample sheets in an oven at approximately 70 C to dry out all volatiles in the liquid cosmetics.

Spray a thick layer of water soluble polymers on the liquid cosmetic drops to protect the cosmetic from transferring to other surfaces.

Heat sample sheet again in oven to dry the sprayed-on polymers.

### Example 2

Use of Cosmetic Sample from Sampler Sheet

Consumer selects the desired cosmetic sample for use or testing. If sample sheet is perforated, the desired sample may be torn from the sheet and the remaining samples on the sample sheet retained for later use. The desired sample is then wetted with water such that the solid on the sample sheet becomes viscous. The consumer may then use her fingers to wipe the cosmetic from the sample sheet and apply to the face or desired area of the skin. The addition of more or less water to the cosmetic sample can affect the degree of color intensity or coverage of certain cosmetic samples.

All percentages are by weight, based on the total weight of the composition, unless otherwise indicated.

All references including patent applications and publications cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A personal care product comprising: (i) a substrate formed of a sheet of plastic or paper, (ii) a layer of a solid water-soluble polymeric matrix forming a continuous layer over said substrate, (iii) a topical formulation disposed between said substrate and said water-soluble polymeric matrix or dispersed throughout said water-soluble polymeric matrix, and (iv) optionally, a protective mask covering said water-soluble polymeric matrix layer; wherein said topical formulation is capable of reconstitution with a suitable volume of water to produce a liquid composition for application to a human integument.

2. The personal care product according to claim 1, wherein said solid water-soluble polymeric matrix comprises a water-soluble polymer selected from the group consisting of polysaccharide polymers, acrylic polymers or copolymers, vinyl alcohol polymers and copolymers, acrylamide polymers and copolymers, polyalkylene oxides, natural hydrocolloids, and combinations thereof.

3. The personal care product according to claim 2, wherein said solid water-soluble polymeric matrix comprises a polysaccharide polymer.

4. The personal care product according to claim 1, wherein said liquid composition for application to a human integument is a lip gloss.

5. The personal care product according to claim 1, wherein said liquid composition for application to a human integument is a foundation.

6. The personal care product according to claim 1, wherein said topical formulation is disposed between said substrate and said solid water-soluble polymeric matrix.

7. The personal care product according to claim 1, wherein said topical formulation is dispersed throughout said solid water-soluble polymeric matrix.

8. The personal care product according to claim 1, wherein said topical formulation comprises a pigment selected from the group consisting of titanium dioxide, calcium carbonate, silica, talc, mica, iron oxide, zinc oxide, and combinations thereof.

9. The personal care product according to claim 1, wherein said topical formulation comprises a pearl selected from the group consisting of white titanated micas, colored titanated micas, bismuth oxychloride, natural nacre, and combinations thereof.

10. The personal care product according to claim 1, wherein said topical formulation includes a particulate selected from the group consisting of nylon power, polyethylene powder, teflon powder, starch, boron nitride, cross-linked polymethacrylate copolymers powders, polymeric microspheres, silicone resins, and combinations thereof.

11. The personal care product according to claim 1, wherein said topical formulation includes an emulsifier and a non-volatile oil selected from the groups consisting of fatty acid alcohols, fatty acid esters, lanolin, hydrocarbon oils, silicone oils, and mixtures thereof.

12. The personal care product according to claim 1, wherein said topical formulation includes a film former selected from the group consisting of waxes, water-soluble polymeric film formers, and water-insoluble polymeric film formers.

13. The personal care product according to claim 1, wherein said topical formulation includes one or more of an emollient, humectant, gellant, thickener, rheology modifier, and combination thereof.

14. The personal care product according to claim 1, wherein said sheet of plastic or paper is between about 1 and about 20 mils in thickness and said layer of solid water-soluble polymeric is from about 0.1 to about 10 mils in thickness.

15. A cosmetic sample sheet comprising: (i) a substrate formed of a sheet of plastic or paper having a plurality of sampling areas defined thereon, (ii) cosmetic formulations deposited on each of said sampling areas, the cosmetic formulations optionally being different at each sample area; (iii) a layer of a solid water-soluble polymeric matrix forming a continuous layer over each of said sampling areas, and (iv) optionally, a protective mask covering said water-soluble polymeric matrix layer; wherein said cosmetic formulations are capable of reconstitution with a suitable volume of water to produce a liquid cosmetic composition for application to a human integument; and wherein said substrate is perforated or scored to permit each sampling area to be readily separated from the others.

16. The cosmetic sample sheet according to claim 15, wherein said cosmetic formulations are characterized by at least one different optical attribute at each sampling area.

17. The cosmetic sample sheet according to claim 16, wherein said cosmetic formulations are characterized by a different hue at each sampling area.

18. The cosmetic sample sheet according to claim 15, wherein said liquid cosmetic composition for application to a human integument is a foundation.

19. A magazine or catalogue insert comprising the cosmetic sample sheet according to claim 15.

20. A method of making a cosmetic sample sheet comprising (i) providing a substrate formed of a sheet of plastic or paper having a plurality of sampling areas defined thereon, (ii) depositing cosmetic formulations on each of said sampling areas, the cosmetic formulations comprising volatile constituents and optionally being different at each sample area; (iii) heating the substrate to remove the volatile components; (iv) spraying a solution or dispersion of water-soluble polymer over said sampling areas; and (v) drying the sample sheet to solidify said water-soluble polymer into a coating layer covering said cosmetic formulations.

21. The method according to claim 20, wherein said substrate is perforated or scored to permit each sampling area to be readily separated from the others.
